(19) United States
(12) Patent Application Publication
(10) Pub. No.: US 2023/0014912 A1
(43) Pub. Date: Jan. 19, 2023

(54) SYRINGE ASSEMBLY AND LIQUID-MEDICINE ADMINISTRATION DEVICE

(71) Applicant: TERUMO KABUSHIKI KAISHA, Tokyo (JP)

(72) Inventor: Junichi OGAWA, Kofu-shi (JP)

(73) Assignee: TERUMO KABUSHIKI KAISHA, Tokyo (JP)

(21) Appl. No.: 17/946,809

(22) Filed: Sep. 16, 2022

Related U.S. Application Data
(63) Continuation of application No. PCT/JP2021/008872, filed on Mar. 8, 2021.

(30) Foreign Application Priority Data
Mar. 17, 2020 (JP) 2020-045900

(51) Int. Cl. A61M 5/31; A61M 5/28

(52) U.S. Cl. CPC A61M 5/3134; A61M 5/285; A61M 2005/3106

(57) ABSTRACT

Provided are a syringe assembly and a liquid-medicine administration device configured to suppress a variation in a pressure limit of a seal member. In the syringe assembly, the seal member is pressed and held between a pressing surface of a cap and a sealing surface of a distal nozzle portion of a syringe. The seal member is pressed and held in the axial direction in a pressing space over the whole region of an opposing portion between the pressing surface and the sealing surface, and a gap between the pressing surface and the sealing surface in the pressing space is narrowest on the inner peripheral side.

### CROSS-REFERENCES TO RELATED APPLICATIONS

This application is a continuation of International Application No. PCT/JP2021/008872 filed on Mar. 8, 2021, which claims priority to Japanese Patent Application No. 2020-045900 filed on Mar. 17, 2022, the entire content of both of which is incorporated herein by reference.

### TECHNOLOGICAL FIELD

The present invention generally relates to a syringe assembly sealing a liquid medicine and a liquid-medicine administration device.

### BACKGROUND DISCUSSION

Conventionally, there is proposed a liquid-medicine administration device of a syringe pump type in which a liquid medicine charged in a cylindrical body is administered into a living body under pressing action of a plunger (WO 2019/182031 A). The liquid-medicine administration device includes a syringe assembly filled with a liquid medicine. A syringe assembly includes a syringe having a distal nozzle portion, a cap fixed to the distal nozzle portion, and a seal member arranged between the cap and the distal nozzle portion and made of an elastic body that seals an opening of the distal nozzle portion.

### SUMMARY

In an assembly step of the syringe assembly, there is a problem that the compression of the seal member becomes non-uniform in the circumferential direction, an internal pressure (pressure limit) at which liquid leakage occurs varies, and the syringe assembly having an extremely low pressure limit is formed.

In the cap, alignment of an axis of the cap and an axis of the syringe coaxially and fitting the cap toward the distal nozzle portion is performed by a mounting device. Meanwhile, it has been found that the axis of the cap is sometimes inclined with respect to the axis of the syringe when the cap is assembled, a crushing load on the seal member by the distal nozzle portion varies to cause a variation in the pressure limit.

The syringe assembly and a liquid-medicine administration device disclosed here are able to suppress a variation in a pressure limit of a seal member.

An aspect of the following disclosure relates to a syringe assembly including: a syringe that includes a distal nozzle portion and a lumen; a cap fixed to the distal nozzle portion; and a seal member arranged between the cap and the distal nozzle portion and made of an elastic body that seals the distal nozzle portion, the seal member including a distal end facing towards the cap and a proximal end facing away from the cap. The cap includes a pressing surface that is in surface contact with the distal end of the seal member and presses the seal member toward a proximal side. The distal nozzle portion includes a distal end at which is located a sealing surface, with the sealing surface of the distal nozzle portion opposing the pressing surface and being in surface contact with the proximal end of the seal member, and the distal nozzle portion including a distal opening on an inner peripheral side of the sealing surface, wherein the distal opening of the distal nozzle portion is in communication with the lumen in the syringe. The seal member closes the distal opening and is pressed and held in an axial direction in a pressing space over a whole region of an opposing portion between the pressing surface and the sealing surface, and a gap between the pressing surface and the sealing surface in the pressing space is narrowest on an inner peripheral side.

According to another aspect, a syringe assembly comprises: a syringe that includes a distal nozzle portion, with the syringe including a lumen extending throughout the distal nozzle portion and opening to a distal opening at a distal end of the distal nozzle portion, and the distal end of the distal nozzle portion including a sealing surface that surrounds the distal opening at the distal end of the distal nozzle portion; a cap fixed to the distal nozzle portion, with the cap including a pressing surface that faces toward the sealing surface at the distal end of the distal nozzle portion; and a seal member made of elastic material and positioned between the sealing surface of the distal nozzle portion and the pressing surface of the cap. The seal member includes both a proximal end surface in direct contact with a contact portion of the sealing surface of the distal nozzle portion and a distal end surface in direct contact with a contact portion of the pressing surface of the cap so that the seal member closes and seals the distal opening at the distal end of the distal nozzle portion. The seal member is pressed and held in an axial direction in a pressing space between the pressing surface and the sealing surface. The contact portion of the pressing surface and the contact portion of the sealing surface are configured so that a first straight line distance between the contact portion of the pressing surface and the contact portion of the sealing surface is less than a second straight line distance between the contact portion of the pressing surface and the contact portion of the sealing surface. The first straight line distance and the second straight line distance both are parallel to the central axis of the distal nozzle portion, with the first straight line distance being spaced from the second straight line distance, and the first straight line distance being located closer to the central axis of the distal nozzle portion than the second straight line distance.

Another aspect relates to a liquid-medicine administration device including: the syringe assembly according to the above aspect; a gasket arranged to be slidable inside the syringe; a plunger assembly capable of pressing the gasket in a distal direction, and a drive mechanism driving the plunger assembly.

According to the syringe assembly and the liquid-medicine administration device of the above aspects, the crushing load on the seal member by the distal nozzle portion is made uniform, and the variation in the pressure limit is suppressed.

### BRIEF DESCRIPTION OF THE DRAWINGS

FIG. 1 is a schematic diagram of a liquid-medicine administration device including a syringe assembly according to a first embodiment.

FIG. 2 is a perspective view of the liquid-medicine administration device of FIG. 1 from which a housing has been removed.

FIG. 3 is a cross-sectional view of the distal side of the syringe assembly of FIG. 2.

FIG. 4 is a cross-sectional view of the distal side of a syringe assembly according to a comparative example.

FIG. 5 is a cross-sectional view illustrating action of the syringe assembly of FIG. 3.

FIG. 6 is a graph illustrating a measurement result of a pressure limit of the syringe assembly according to Experimental Example 1 (the comparative example).

FIG. 7 is a graph illustrating a measurement result of a pressure limit of the syringe assembly according to Experimental Example 2 (the first embodiment).

FIG. 8 is a cross-sectional view of the distal side of a syringe assembly according to a second embodiment.

FIG. 9 is a cross-sectional view illustrating action of the syringe assembly of FIG. 8.

FIG. 10 is a cross-sectional view of a distal side of a syringe assembly according to a third embodiment.

FIG. 11 is a cross-sectional view illustrating action of the syringe assembly of FIG. 10.

FIG. 12 is a cross-sectional view of a distal side of a syringe assembly according to a fourth embodiment.

FIG. 13 is a cross-sectional view illustrating action of the syringe assembly of FIG. 12.

### DETAILED DESCRIPTION

Hereinafter, preferred embodiments regarding syringe assemblies 12A, 12B, 12C, and 12D and a liquid-medicine administration device 10 will be described in detail with reference to the accompanying drawings, it being understood that the disclosed embodiments represent examples of the new syringe assembly and liquid-medicine administration device disclosed here. In the second to fourth embodiments, features and aspects of the syringe assemblies and liquid-medicine administration device that are similar to features and aspects that have already been described are denoted by the same reference signs, and a detailed description of such features and aspects will not be repeated.

### First Embodiment

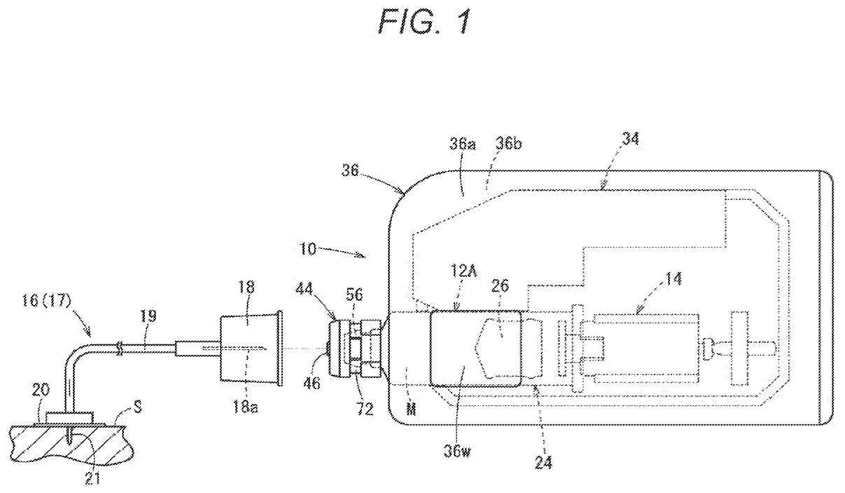

The liquid-medicine administration device 10 illustrated in FIG. 1 is used to administer a liquid medicine M into a living body. The liquid-medicine administration device 10 continuously administers the liquid medicine M charged in (located in) the syringe assembly 12A into the living body under pressing action of a plunger assembly 14 for a relatively long time (for example, about several minutes to several hours). The liquid-medicine administration device 10 may intermittently administer the liquid medicine M into the living body. Examples of the liquid medicine M include a protein preparation, a narcotic analgesic, a diuretic, and the like.

As illustrated in FIG. 1, when the liquid-medicine administration device 10 is used, for example, a patch-type needle-attached tube 17 is connected as an administration tool 16. The liquid medicine M discharged from the syringe assembly 12A is injected into the body of a patient via the needle-attached tube 17. The needle-attached tube 17 includes: a connector 18 that can be connected to a distal nozzle portion 56 of the syringe assembly 12A; a liquid supply tube 19 having one end connected to the connector 18 and having flexibility; a patch portion 20 that is connected to the other end of the liquid supply tube 19 and is configured to be stuck to skin S; and a puncture needle 21 protruding from the patch portion 20. The puncture needle 21 substantially perpendicularly punctures the skin S. The puncture needle 21 may alternatively be one that obliquely punctures the skin S.

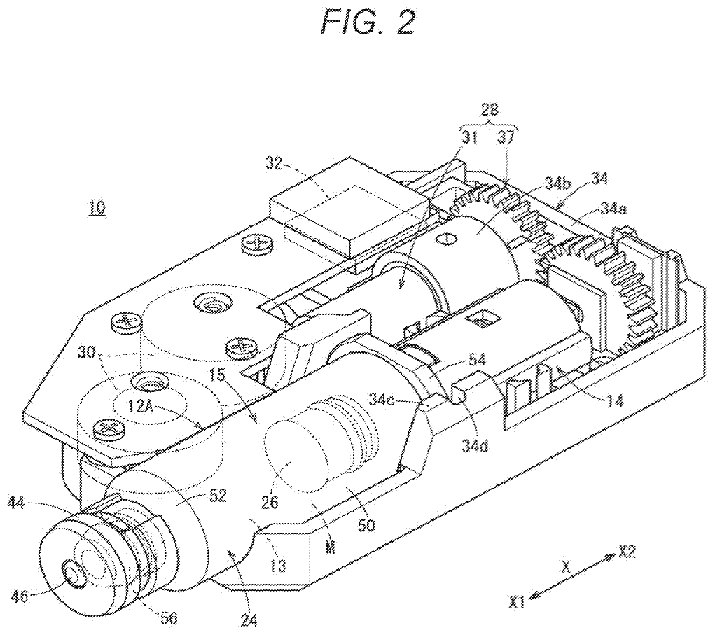

As illustrated in FIG. 1 or 2, the liquid-medicine administration device 10 includes: the syringe assembly 12A having a syringe 24 filled with the liquid medicine M; a gasket 26 arranged to be slidable inside the syringe 24; the plunger assembly 14 that is movable in the axial direction (arrow X direction) and can press the gasket 26 in the distal direction (arrow X1 direction); a drive mechanism 28 that drives the plunger assembly 14; a battery 30 that supplies electric power necessary for the operation of the liquid-medicine administration device 10; a control unit 32 that controls the drive mechanism 28; a chassis structure 34 that supports the syringe assembly 12A, the plunger assembly 14, and the drive mechanism 28; and a housing 36 that houses these.

As illustrated in FIG. 2, the syringe assembly 12A includes: the syringe 24 having the distal nozzle portion 56; a cap 44 mounted on the distal nozzle portion 56 of the syringe 24; and a seal member 46 arranged between the distal nozzle portion 56 and the cap 44.

The syringe 24 is formed in a hollow cylindrical shape. Specifically, the syringe 24 includes a barrel portion 50 having a lumen 13 that can be filled with the liquid medicine M, and a flange portion 54 protruding outward from an outer peripheral surface of the barrel portion 50. The inside of the syringe 24 is filled with the liquid medicine M in advance. The syringe 24 may be made of a transparent material.

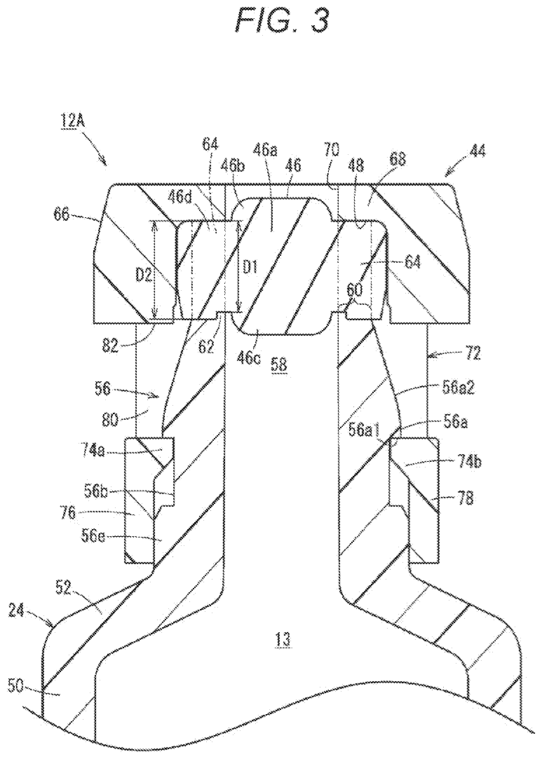

As illustrated in FIG. 3, a distal portion of the distal nozzle portion 56 is provided with an engagement projecting portion 56a. The engagement projecting portion 56a is formed in an annular shape that protrudes radially outward and extends to around the distal nozzle portion in the circumferential direction in an annular shape. The engagement projecting portion 56a includes: a locking surface 56a1 on which claw portions 74a and 74b, which will be described later, of the cap 44 are locked; and an inclined surface 56a2 which is formed on the distal side of the locking surface 56a1 and reduced in diameter in the distal direction. The locking surface 56a1 is a flat surface perpendicular to an axis of the distal nozzle portion 56. On an outer peripheral surface of the distal nozzle portion 56, an annular groove 56b that is recessed radially inward is formed on the proximal side of the engagement projecting portion 56a.

An anti-rattling projecting portion 56e that prevents rattling of the cap 44 is provided on the outer periphery of a proximal portion of the distal nozzle portion 56. The anti-rattling projecting portion 56e is formed in an annular shape that bulges radially outward and extends t in the circumferential direction in an annular shape. An outer peripheral surface of the anti-rattling projecting portion 56e extends along the axis of the distal nozzle portion 56. A proximal end of the anti-rattling projecting portion 56e is connected to a distal end of the shoulder portion 52.

A sealing surface 60 perpendicular to the axial direction is formed at a distal end of the distal nozzle portion 56. The sealing surface 60 is formed in a circular ring shape (annular shape) on the outer peripheral side of a distal opening 58. Since the substantially entire radial region of the sealing surface 60 is in surface contact with the seal member 46, high sealing performance (pressure resistance) is exhibited, and liquid leakage can be prevented even if a relatively high pressure of the lumen 13 acts. The sealing surface 60 can have an inner diameter of about 0.5 to 5.0 mm, and an outer diameter of about 2.0 to 9.0 mm.

A ring-shaped protrusion 62 protruding toward the axially distal side (a pressing surface 48) is formed on the inner peripheral side of the sealing surface 60. The ring-shaped protrusion 62 is formed in an annular shape along an inner peripheral portion of the sealing surface 60. A protruding height of the ring-shaped protrusion 62 is formed to such an extent as not to hinder the surface contact between an outer peripheral portion 46d of the seal member 46 and the sealing surface 60. The ring-shaped protrusion 62 prevents movement of a wall of the seal member 46 from moving when the cap 44 is mounted. As dimensions of the ring-shaped protrusion 62 exhibiting such a function, for example, a protruding height in the axial direction can be about 0.05 to 1.5 mm and a dimension in the radial direction (width direction) can be about 0.05 to 1.5 mm.

The seal member 46 is punctured by a needle 18a provided in the connector 18 when the connector 18 illustrated in FIG. 1 is connected to the distal nozzle portion 56. The seal member 46 is made of an elastic resin material such as a rubber material or an elastomer material formed in a plate shape (plate-shaped seal member). The seal member 46 is fixed to the distal nozzle portion 56 of the syringe assembly 12A by the cap 44 to seal the distal opening 58 of the distal nozzle portion 56. The seal member 46 is held in a state of being elastically compressed in the axial direction between the sealing surface 60 of the distal nozzle portion 56 and the pressing surface 48 of the cap 44. The seal member 46 is formed in a disc shape.

The seal member 46 includes: a seal body portion 46a forming a central portion in the thickness direction; a distal projecting portion 46b protruding in the distal direction from the distal side of the seal body portion 46a; and a proximal projecting portion 46c protruding in the proximal direction from the proximal side of the seal body portion 46a. The distal projecting portion 46b slightly protrudes in the distal direction from the pressing surface 48 of the cap 44. A surface on the distal side of the distal projecting portion 46b may be located at the same axial position as the pressing surface 48 of the cap 44 or on the proximal side of the pressing surface 48.

The outer peripheral portion 46d of the distal projecting portion 46b and a proximal projecting portion 46c of the seal member 46 is arranged in a pressing space 64 formed between the sealing surface 60 of the distal nozzle portion 56 and the pressing surface 48 of the cap 44. In the pressing space 64, a proximal end of the outer peripheral portion 46d of the seal member 46 is in surface contact and close contact with substantially the entire radial region of the sealing surface 60, thereby exhibiting the pressure resistance. In addition, the outer peripheral portion 46d of the seal member 46 is in surface contact with the pressing surface 48 of the cap 44 on the distal side. Then, the outer peripheral portion 46d is pressed in the axial direction by the pressing surface 48 and the sealing surface 60.

The pressing space 64 is a space between the sealing surface 60 of the distal nozzle portion 56 and the pressing surface 48 of the cap 44 at a portion opposing the sealing surface, and is formed in a circular ring shape on the outer peripheral side of the distal opening 58 of the distal nozzle portion 56. Since the ring-shaped protrusion 62 is formed along the inner peripheral side of the sealing surface 60 in the present embodiment, a width D1 on the inner peripheral side is narrower than a width D2 on the outer peripheral side regarding a width D (gap) of the pressing space 64 in the axial direction. That is, the width D of the pressing space 64 in the axial direction is narrowest on the inner peripheral side. As shown in FIG. 3, the straight line distance D1 (parallel to the central axis of the distal nozzle portion 56 and the central axis of the seal member 46) between the part of the sealing surface 60 at which the protrusion 62 is formed and the pressing surface 48 of the cap 44 is less than the straight line distance D2 (parallel to the central axis of the distal nozzle portion 56 and the central axis of the seal member 46) between a part of the sealing surface 60 outwardly of the protrusion 62 and the pressing surface 48 of the cap 44. As also shown in FIG. 3, a contact portion of the pressing surface 48 of the cap 44 directly contacts the distal end surface of the seal member 46, and a contact portion of the sealing surface 60 of the distal nozzle portion 56 directly contacts the distal end surface of the seal member 46.

The cap 44 includes a base portion 66 provided on the distal side of the cap 44, and a mounting portion 72 which has a cylindrical shape, extends from the base portion 66 in the proximal direction along an axis of the cap 44, and covers the outside of the distal nozzle portion 56. The pressing surface 48 that is in surface contact with the seal member 46 is formed on the proximal side of the base portion 66. The pressing surface 48 is formed in a circular ring shape (annular shape) around a through-hole 70 at the center. The through-hole 70 is formed in a central portion of the base portion 66, penetrates the base portion in the axial direction and exposes the distal projecting portion 46b of the seal member 46.

The through-hole 70 is a hole formed in a circular shape when viewed from the axial direction, and is coaxial with the cap 44. The through-hole 70 is formed in a distal wall 68 of the base portion 66. The distal projecting portion 46b of the seal member 46 is inserted into or positioned in the through-hole 70.

The mounting portion 72 includes claw portions 74a and 74b, a first column portion 76, and a second column portion 78. The two claw portions 74a and 74b oppose each other across the central axis of the cap 44, and are located at positions spaced apart from the base portion 66 to the proximal side (i.e., the two claw portions 74a and 74b are proximal of the base portion 66). The claw portions 74a and 74b protrude inward from an inner peripheral surface of the mounting portion 72. The cap 44 is mounted to the distal nozzle portion 56 as the two claw portions 74a and 74b engage with the engagement projecting portion 56a of the distal nozzle portion 56. That is, the two claw portions 74a and 74b engage with the proximal end (locking surface 56a1) of the engagement projecting portion 56a of the distal nozzle portion 56, thereby preventing the cap 44 from coming off from the distal nozzle portion 56. Each of the two claw portions 74a and 74b extends in an arc shape in the circumferential direction along the inner peripheral surface of the mounting portion 72. That is, each of the claw portions 74a, 74b has an arc-shaped circumferential extent so that they each extend less than an entirety of the circumferential extent of the mounting portion 72.

The first column portion 76 is provided on the outer side of one claw portion 74a in the circumferential direction of the mounting portion 72, and extends to the proximal side along the axis of the cap 44. The second column portion 78 is provided on the outer side of the other claw portion 74b in the circumferential direction of the mounting portion 72, and extends to the proximal side along the axis of the cap 44. Inner peripheral surfaces of the first column portion 76 and the second column portion 78 abut on the anti-rattling projecting portion 56e of the distal nozzle portion 56, thereby preventing the cap 44 from being inclined with respect to the distal nozzle portion 56. The first column portion 76 and the second column portion 78 are integrally connected to the base portion 66 via the side wall portion 80. Two side holes 82 are formed on the distal side of the side wall portion 80. The side holes 82 penetrate the side wall portion 80 in the radial direction.

In FIG. 2, the gasket 26 liquid-tightly seals the proximal side of the lumen 13 of the syringe 24. In an initial state of the liquid-medicine administration device 10, the gasket 26 is located on the distal side of a proximal end of the syringe 24. An outer portion of the gasket 26 is in close contact with an inner peripheral surface of the syringe 24 (barrel portion 50) in a liquid-tight manner. The syringe assembly 12A, the liquid medicine M, and the gasket 26 form a prefilled syringe 15.

The plunger assembly 14 is configured to advance the gasket 26 inside the syringe 24 and push out the liquid medicine M from the syringe assembly 12A. In the initial state of the liquid-medicine administration device 10, the distal side of the plunger assembly 14 is inserted into the proximal side of the syringe 24. The drive mechanism 28 includes: a motor 31 which is driven and controlled under control of the control unit 32 using the battery 30 as a power source; and a drive gear 37 fixed to an output shaft of the motor 31.

The chassis structure 34 is arranged inside the housing 36 (see FIG. 1). The syringe assembly 12A, the drive mechanism 28, and the plunger assembly 14 are fixed to predetermined positions of the chassis structure 34, respectively. The chassis structure 34 includes a chassis body member 34a and a motor holding member 34b that is fixed to the chassis body member 34a and holds the motor 31 against the chassis body member 34a.

The chassis body member 34a has a flange holding portion 34c that protrudes upward and holds the flange portion 54 of the syringe 24. The flange holding portion 34c is provided with a holding groove 34d into which the flange portion 54 is inserted.

In FIG. 1, the housing 36 is a hollow member configured to house the syringe assembly 12A, the gasket 26, the plunger assembly 14, the drive mechanism 28, the battery 30, the control unit 32, and the chassis structure 34 described above. The distal nozzle portion 56 of the syringe assembly 12A protrudes from the housing 36, and the cap 44 is exposed to the outside. A window portion 36w made of a transparent material is provided on an upper surface 36a of the housing 36.

The liquid-medicine administration device 10 can be configured as a patch-type device that is used by being stuck to the skin S of the patient, for example. In the case of such a patch-type device, a sheet-shaped sticking portion (adhesive portion) that can be stuck to the skin S is provided on a bottom surface 36b of the housing 36. In the initial state of the liquid-medicine administration device 10, a peelable protective sheet is stuck to a sticking surface of the sticking portion.

The liquid-medicine administration device 10 may be provided with a mounting tool, such as a hook and a clip, on the bottom surface 36b of the housing 36 and mounted by a method of being hooked on clothes of the patient (for example, a waist portion of pants or the like).

Next, the action (assembly) of the syringe assembly 12A will be described with reference to a comparative example.

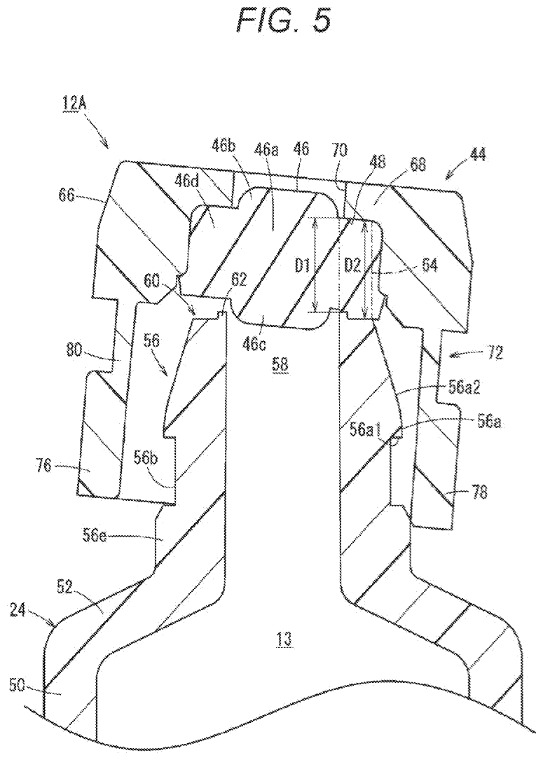

In the assembly step of the syringe assembly 12A, a syringe preparation step, a seal member preparation step, a cap preparation step, a cap assembly step, and a cap mounting step (see FIG. 5) are sequentially performed.

In the syringe preparation, the syringe 24 including the distal nozzle portion 56 having the engagement projecting portion 56a provided on the outer peripheral surface and the distal opening 58 is prepared.

In the seal member preparation, the plate-shaped seal member 46 having elasticity is prepared.

In the cap preparation, the cap 44 including the base portion 66, made of a hard resin material and provided at the distal end, and the cylindrical mounting portion 72, which extends from the base portion 66 in the proximal direction along the axis of the cap 44, is prepared.

In the cap assembly, the seal member 46 is inserted into the inner peripheral side of the mounting portion 72 of the cap 44, and the seal member 46 is in surface contact with the pressing surface 48. In addition, the distal projecting portion 46b of the seal member 46 is inserted into or positioned in the through-hole 70.

In the cap mounting, the distal nozzle portion 56 is inserted into the mounting portion 72 of the cap 44 to which the seal member 46 is attached. Then, the cap 44 is pushed to the proximal side or in the proximal direction (downward in FIG. 5) until the two claw portions 74a and 74b of the mounting portion 72 pass over the engagement projecting portion 56a of the distal nozzle portion 56. Then, the distal opening 58 of the distal nozzle portion 56 is sealed with the seal member 46.

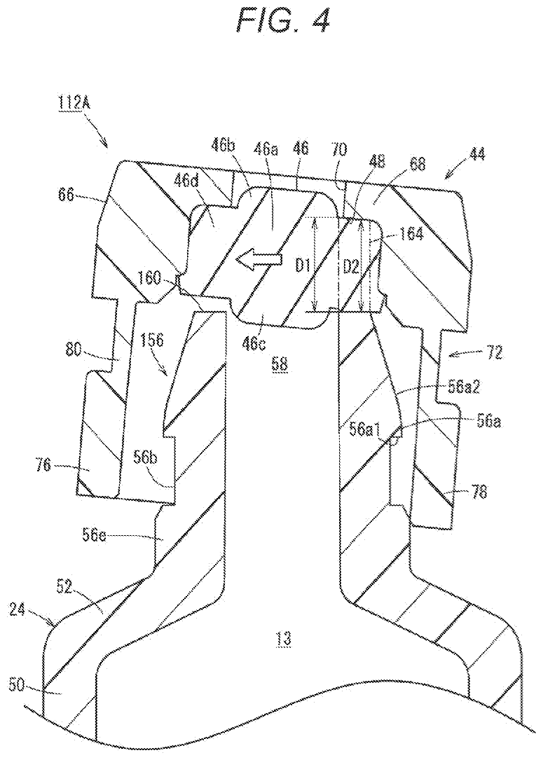

The cap mounting is performed by press-fitting the cap 44 toward the distal nozzle portion 56 while gripping the cap 44 and the syringe 24 with a chuck of the mounting device. In a state in FIG. 5 where the cap mounting is completed, the inner peripheral surface of the mounting portion 72 of the cap 44 abuts on the anti-rattling projecting portion 56e of the distal nozzle portion 56, and the cap 44 is assembled without being inclined. However, in the middle of pushing the cap 44 into the distal nozzle portion 56, the cap 44 may be inclined with respect to the distal nozzle portion 56 as illustrated in FIGS. 4 and 5 due to various factors.

As illustrated in FIG. 4, in a syringe assembly 112A of the comparative example, no ring-shaped protrusion 62 is formed on a sealing surface 160 of a distal nozzle portion 156. In the process of pushing a cap 44, a seal member 46 is in surface contact with a pressing surface 48 of the cap 44 and the sealing surface 160 of the distal nozzle portion 156 to form a pressing space 164. When the cap 44 is inclined in the case of the comparative example, the pressing space 164 has a shape that is open in a V shape so as to gradually expand toward the inner peripheral side as a width D1 in the axial direction on the inner peripheral side becomes larger than a width D2 in the axial direction on the outer peripheral side. Therefore, in the process of pushing the cap 44 to the proximal side, a wall of the seal member 46 moves so as to escape from the pressing space 164 toward a non-pressed site.

As a result, a compressive load on the sealing surface 160 varies in the circumferential direction of the seal member 46, and a pressure limit decreases. That is, in the seal member 46, a wall thickness of a portion that first abuts on the sealing surface 160 is insufficient, and the sealing performance between the sealing surface 160 and the seal member 46 deteriorates in the state after the cap 44 is mounted.

On the other hand, in the syringe assembly 12A of the present embodiment, the ring-shaped protrusion 62 is formed on the inner peripheral portion of the sealing surface 60 of the distal nozzle portion 56 as illustrated in FIG. 5. In the process of pushing the cap 44, the seal member 46 is in surface contact with the pressing surface 48 of the cap 44 and the sealing surface 60 of the distal nozzle portion 56 to form the pressing space 64. Since the ring-shaped protrusion 62 is provided as illustrated in the drawing, the width D1 in the axial direction on the inner peripheral side of the pressing space 64 is smaller than the width D2 in the axial direction on the outer peripheral side thereof, and the width D1 in the axial direction of the inner peripheral portion of the pressing space 64 is the narrowest. As a result, the movement of the wall of the seal member 46 is prevented. As a result, a compressive load on the sealing surface 60 becomes uniform in the circumferential direction of the seal member 46, and the pressure limit can be improved.

Next, a description will be given regarding results obtained by actually preparing the syringe assembly 112A of the comparative example (Experimental Example 1) and the syringe assembly 12A of the present embodiment (Experimental Example 2) and measuring a pressure limit at which liquid leakage occurs when the gasket 26 is pushed.

In Experimental Example 1, five samples in which water was sealed as a test liquid in the syringe assembly 112A of Comparative Example 1 illustrated in FIG. 4 were prepared. Then, a gasket 26 was pushed by a test plunger, and a displacement (mm) of the test plunger and a test force (N), which is an input load with respect to the test plunger, were measured.

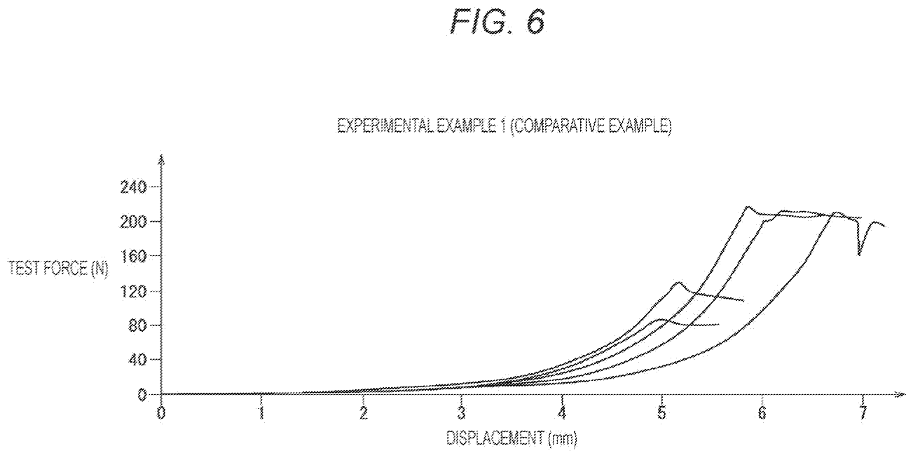

FIG. 6 illustrates measurement results of Experimental Example 1. In FIG. 6, the vertical axis represents the test force (N), and the horizontal axis represents the displacement (mm) of the test plunger. The test force reflects the internal pressure of a syringe 24 and increases as the displacement of the test plunger increases. When liquid leakage occurs, the test force does not increase and becomes constant even if the displacement of the test plunger increases. Therefore, the maximum value of the test force reflects the pressure limit by the seal member 46 in FIG. 6.

As illustrated in the drawing, in Experimental Example 1, the maximum value of the test force ranges from 80 to 200 N with a relatively large variation. Regarding two samples, the pressure limit was an extremely low value between 80 and 120 N.

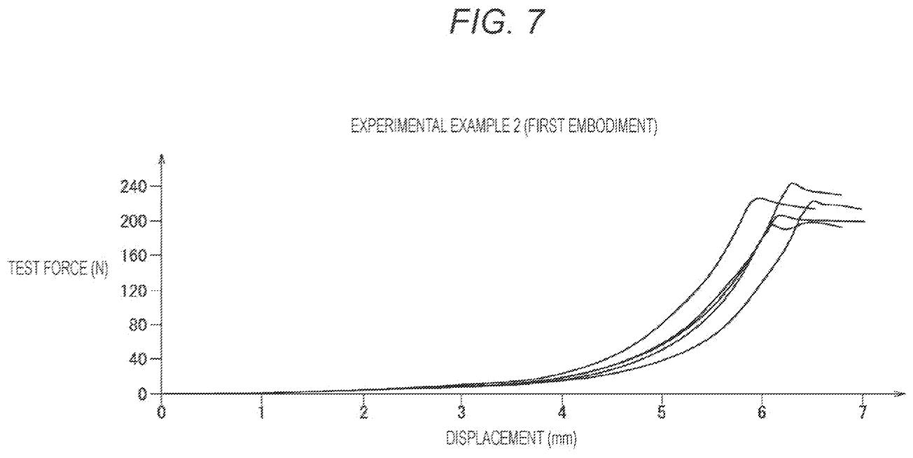

On the other hand, in Experimental Example 2, five samples in which water was sealed in the syringe assembly 12A of the present embodiment illustrated in FIG. 3 were prepared, and the pressure limit was evaluated by the same method as in Experimental Example 1. FIG. 7 illustrates measurement results of Experimental Example 2.

As illustrated in FIG. 7, in Experimental Example 2 (the present embodiment), the maximum value of the test force fell within the range of 200 to 240 N, and it has been confirmed that a variation in the pressure limit was suppressed, and there is no sample having an extremely low pressure limit.

The syringe assembly 12A and the liquid-medicine administration device 10 of the present embodiment have the following effects.

The syringe assembly 12A of the present embodiment includes the syringe 24 having the distal nozzle portion 56, the cap 44 fixed to the distal nozzle portion 56, and the seal member 46 arranged between the cap 44 and the distal nozzle portion 56 and made of the elastic body that seals the distal nozzle portion 56. In the syringe assembly 12A, the cap 44 has the pressing surface 48 that is in surface contact with the distal end of the seal member 46 and presses the seal member 46 to the proximal side, the distal nozzle portion 56 has the sealing surface 60, which opposes the pressing surface 48 and is in surface contact with the proximal end of the seal member 46, at the distal end and the distal opening 58 which is formed on the inner peripheral side of the sealing surface 60 and communicates with the lumen 13 of the syringe 24, the seal member 46 closes the distal opening 58 and is pressed and held in the axial direction in the pressing space 64 over a whole region of the opposing portion between the pressing surface 48 and the sealing surface 60, and the gap between the pressing surface 48 and the sealing surface 60 in the pressing space 64 is narrowest on the inner peripheral side.

According to the above configuration, it is possible to prevent the movement of the wall of the seal member 46 even when the cap 44 is inclined with respect to the distal nozzle portion 56 at the time of mounting the cap 44 on the distal nozzle portion 56. As a result, it is possible to suppress the variation in the compressive load of the seal member 46 in the circumferential direction and to prevent the decrease in the pressure limit caused by the seal member 46.

In the syringe assembly 12A, the sealing surface 60 may be provided with the ring-shaped protrusion 62 which is formed in the annular shape along the inner peripheral side and protrudes toward the pressing surface 48. Since the ring-shaped protrusion 62 is provided on the inner peripheral side in this manner, the gap between the pressing surface 48 and the sealing surface 60 in the pressing space 64 can be made narrowest on the inner peripheral side, and the movement of the wall of the seal member 46 can be prevented.

In addition, the liquid-medicine administration device 10 of the present embodiment includes: the syringe assembly 12A that includes the syringe 24 having the distal nozzle portion 56, the cap 44 fixed to the distal nozzle portion 56; the seal member 46 arranged between the cap 44 and the distal nozzle portion 56 and made of the elastic body that seals the distal nozzle portion 56, and the cap 44 having the pressing surface 48 that is in surface contact with the distal end of the seal member 46 and presses the seal member 46 to the proximal side, the distal nozzle portion 56 having the sealing surface 60, which opposes the pressing surface 48 and is in surface contact with the proximal end of the seal member 46, at the distal end and the distal opening 58 which is formed on the inner peripheral side of the sealing surface 60 and communicates with the lumen 13 of the syringe 24, the seal member 46 closing the distal opening 58 and being pressed and held in the axial direction in the pressing space 64 over the whole region of the opposing portion between the pressing surface 48 and the sealing surface 60, and a gap between the pressing surface 48 and the sealing surface 60 in the pressing space 64 being narrowest on the inner peripheral side; the gasket 26 arranged to be slidable inside the syringe 24; the plunger assembly 14 capable of pressing the gasket 26 in the distal direction; and the drive mechanism 28 driving the plunger assembly 14.

According to the liquid-medicine administration device 10 having the above configuration, the variation in the pressure limit of the seal member 46 is suppressed, and thus, the liquid leakage hardly occurs.

### Second Embodiment

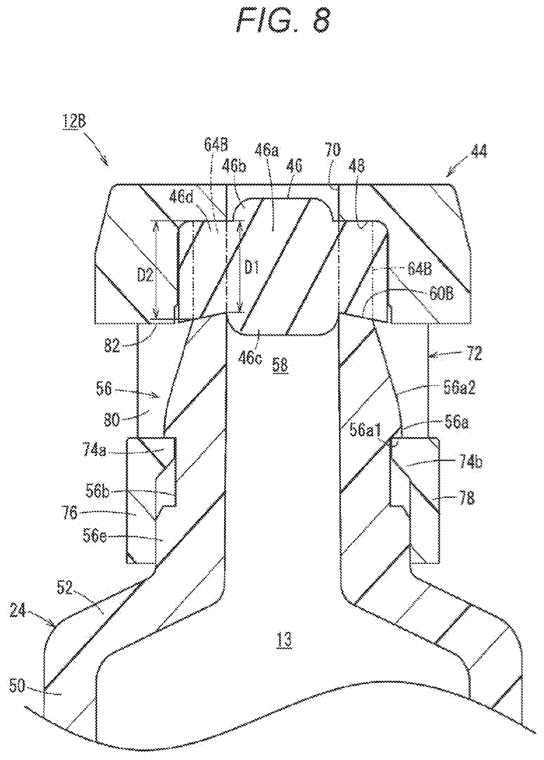

As illustrated in FIG. 8, the syringe assembly 12B of the present embodiment is configured such that a sealing surface 60B of the distal nozzle portion 56 is an inclined surface inclined with respect to the pressing surface 48 of the cap 44. In the sealing surface 60B, the inner peripheral side adjacent to the distal opening 58 protrudes toward the pressing surface 48 the most. That is, the sealing surface 60B is configured as the inclined surface inclined so as to gradually protrude or project toward the pressing surface 48 from the outer peripheral side to the inner peripheral side. The inner peripheral side of the sealing surface 60B thus protrudes distally beyond or distally farther then the outer peripheral side of the sealing surface 60B In this manner, a width D1 in the axial direction on the inner peripheral side of a pressing space 64B, which presses the outer peripheral portion 46d of the seal member 46 in the axial direction, is the narrowest even in the syringe assembly 12B. As shown in FIG. 8, the straight line distance D1 (parallel to the central axis of the distal nozzle portion 56 and the central axis of the seal member 46) between the part of the inclined sealing surface 60B that protrudes axially farthest toward the cap 44 and the pressing surface 48 of the cap 44 is less than the straight line distance D2 (parallel to the central axis of the distal nozzle portion 56 and the central axis of the seal member 46) between a part of the inclined sealing surface 60 that is outwardly of the part protruding axially farthest and the pressing surface 68 of the cap 44.

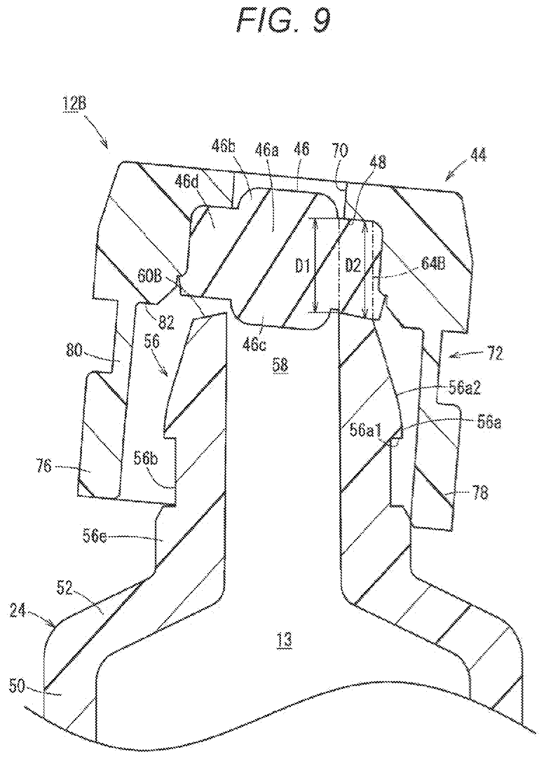

Since the width D1 in the axial direction on the inner peripheral side of the pressing space 64B is the narrowest even when the cap 44 is inclined, movement of a wall of the seal member 46 can be prevented in a cap mounting step as illustrated in FIG. 9. As a result, a pressing load in the circumferential direction of the seal member 46 is made uniform even by the present embodiment so that a variation in a pressure limit can be suppressed.

The syringe assembly 12B of the present embodiment has the following effects.

In the syringe assembly 12B of the present embodiment, the sealing surface 60B is configured as the inclined surface inclined so as to gradually protrude toward the pressing surface from the outer peripheral side to the inner peripheral side. With this configuration, the pressing load in the circumferential direction of the seal member 46 is made uniform so that the variation in the pressure limit can be suppressed.

### Third Embodiment

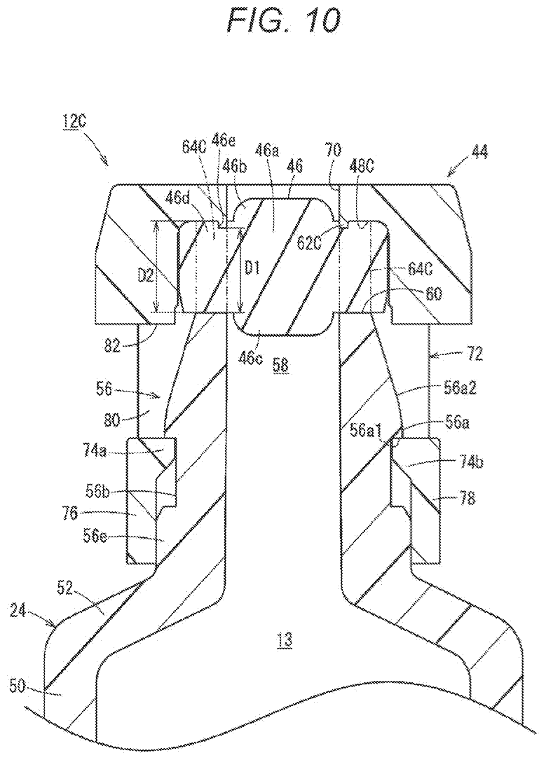

As illustrated in FIG. 10, the syringe assembly 12C of the present embodiment is provided with a ring-shaped protrusion 62C, which protrudes toward the sealing surface 60 of the distal nozzle portion 56 (i.e., in the proximal direction), in an inner peripheral portion of a pressing surface 48C of the cap 44. The ring-shaped protrusion 62C is formed in an annular shape along the through-hole 70 of the cap 44. In the seal member 46, a ring-shaped groove 46e is formed in a portion corresponding to the ring-shaped protrusion 62C, and the ring-shaped protrusion 62C is inserted into the ring-shaped groove 46e.

The sealing surface 60 of the distal nozzle portion 56 is formed as a flat surface perpendicular to the axial direction. Although the ring-shaped protrusion 62 (see FIG. 3) is not formed on the sealing surface 60 in the illustrated example, the present invention is not limited thereto, and the ring-shaped protrusion 62 may be formed on the inner peripheral side of the sealing surface 60.

In the present embodiment, the inner peripheral side where the pressing surface 48C is adjacent to the through-hole 70 protrudes toward the sealing surface 60 the most. Therefore, a width D1 in the axial direction on the inner peripheral side of a pressing space 64C is the narrowest in the pressing space 64C that presses the seal member 46. As shown in FIG. 10, the straight line distance D1 (parallel to the central axis of the distal nozzle portion 56 and the central axis of the seal member 46) between the part of the pressing surface 48C of the cap 44 at which the protrusion 62C is formed and the sealing surface 60 of the distal nozzle portion 56 is less than the straight line distance D2 (parallel to the central axis of the distal nozzle portion 56 and the central axis of the seal member 46) between a part of the pressing surface 48C outwardly of the protrusion 62C and the sealing surface 60 of the distal nozzle portion 56.

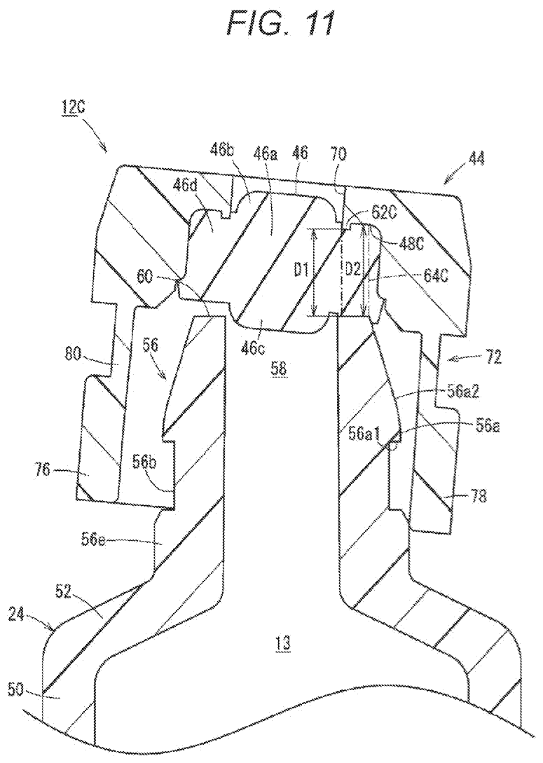

Since the width D1 in the axial direction on the inner peripheral side of the pressing space 64C is the narrowest even when the cap 44 is inclined, movement of a wall of the seal member 46 can be prevented in a cap mounting step as illustrated in FIG. 11. As a result, a pressing load in the circumferential direction of the seal member 46 is made uniform even by the present embodiment so that a variation in a pressure limit can be suppressed.

The syringe assembly 12C of the present embodiment has the following effects.

In the syringe assembly 12C of the present embodiment, the pressing surface 48C is provided with the ring-shaped protrusion 62C which is formed in the annular shape along the inner peripheral side and protrudes toward the sealing surface 60 (i.e., toward the proximal direction). With this configuration, the pressing load in the circumferential direction of the seal member 46 is made uniform so that the variation in the pressure limit can be suppressed.

### Fourth Embodiment

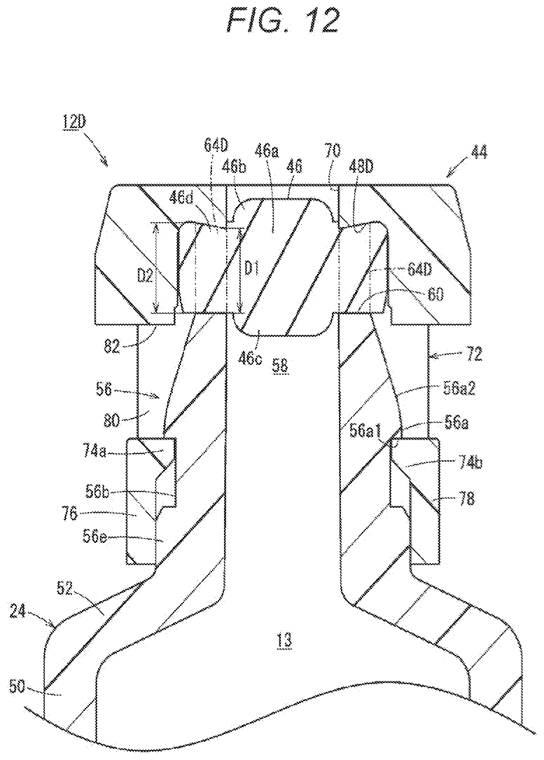

As illustrated in FIG. 12, the syringe assembly 12D of the present embodiment is configured such that a pressing surface 48D of the cap 44 is an inclined surface inclined with respect to the sealing surface 60 of the distal nozzle portion 56. The pressing surface 48D protrudes toward the sealing surface 60 the most on the inner peripheral side adjacent to the through-hole 70. That is, the pressing surface 48D is configured as the inclined surface inclined so as to gradually protrude toward the sealing surface 60 (i.e., in the proximal direction) from the outer peripheral side to the inner peripheral side. In addition, an upper end side of the outer peripheral portion 46d of the seal member 46 of the present embodiment is configured as an inclined surface corresponding to the inclination of the pressing surface 48D.

The sealing surface 60 of the distal nozzle portion 56 is formed as a flat surface perpendicular to the axial direction. Although the ring-shaped protrusion 62 (see FIG. 3) is not formed on the sealing surface 60 in the illustrated example, the present invention is not limited thereto, and the ring-shaped protrusion 62 may be formed on the inner peripheral side of the sealing surface 60.

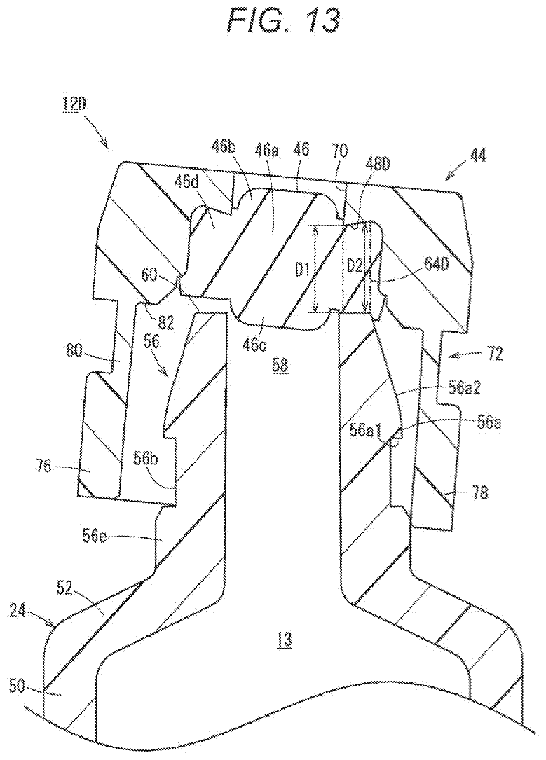

Since a width D1 in the axial direction on the inner peripheral side of the pressing space 64D is the narrowest even when where the cap 44 is inclined, movement of a wall of the seal member 46 can be prevented in a cap mounting step as illustrated in FIG. 13. As a result, a pressing load in the circumferential direction of the seal member 46 is made uniform even by the present embodiment so that a variation in a pressure limit can be suppressed. As shown in FIG. 12, the straight line distance D1 (parallel to the central axis of the distal nozzle portion 56 and the central axis of the seal member 46) between the part of the inclined pressing surface 48D that protrudes axially farthest toward the sealing surface 60 of the distal nozzle portion 56 and the sealing surface 60 is less than the straight line distance D2 (parallel to the central axis of the distal nozzle portion 56 and the central axis of the seal member 46) between a part of the inclined pressing surface 48D that is outwardly of the part protruding axially farthest and the sealing surface 60.

The syringe assembly 12D of the present embodiment has the following effects.

In the syringe assembly 12D, the pressing surface 48D is configured as the inclined surface inclined so as to gradually protrude toward the sealing surface 60 (i.e., in the proximal direction) from the outer peripheral side to the inner peripheral side. With this configuration, the pressing load in the circumferential direction of the seal member 46 is made uniform so that the variation in the pressure limit can be suppressed.

Although the syringe assemblies 12A, 12B, 12C, and 12D, and the liquid-medicine administration device 10 have been described with the preferred embodiments as above, the invention is not limited to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A syringe assembly comprising:
a syringe that includes a distal nozzle portion and a lumen;
a cap fixed to the distal nozzle portion;
a seal member arranged between the cap and the distal nozzle portion and made of an elastic body that seals the distal nozzle portion, the seal member including a distal end facing towards the cap and a proximal end facing away from the cap;
the cap including a pressing surface that is in surface contact with the distal end of the seal member and presses the seal member toward a proximal side,
the distal nozzle portion including a distal end at which is located a sealing surface, the sealing surface of the distal nozzle portion opposing the pressing surface and being in surface contact with the proximal end of the seal member, the distal nozzle portion including a distal opening on an inner peripheral side of the sealing surface, the distal opening of the distal nozzle portion being in communication with the lumen in the syringe;
the seal member closing the distal opening and being pressed and held in an axial direction in a pressing space over a whole region of an opposing portion between the pressing surface and the sealing surface; and
a gap between the pressing surface and the sealing surface in the pressing space is narrowest on an inner peripheral side.

2. The syringe assembly according to claim 1, wherein the sealing surface of the distal nozzle portion includes a ring-shaped protrusion located along an inner peripheral side of the sealing surface and protruding toward the pressing surface.

3. The syringe assembly according to claim 1, wherein the sealing surface is an inclined sealing surface that is inclined to gradually protrude toward the pressing surface of the cap from an outer peripheral side to an inner peripheral side so that a first part of the inclined sealing surface closer to the distal opening of the distal nozzle portion than a second part of the inclined sealing surface is located closer to the pressing surface of the cap than the second part of the inclined sealing surface.

4. The syringe assembly according to claim 1, wherein the pressing surface of the cap includes a ring-shaped protrusion located along an inner peripheral side of the pressing surface and protruding toward the sealing surface.

5. The syringe assembly according to claim 1, wherein the pressing surface is an inclined pressing surface that is inclined to gradually protrude toward the sealing surface of the distal nozzle portion from an outer peripheral side to an inner peripheral side so that a first part of the inclined pressing surface closer to a central axis of the cap than a second part of the inclined pressing surface is located closer to the sealing surface of the distal nozzle portion than the second part of the inclined pressing surface.

6. The syringe assembly according to claim 1, wherein the seal member comprises: a plate-shaped seal body portion that is a central portion of the seal member in a thickness direction of the seal member; a distal projecting portion protruding in a distal direction from a distal side of the seal body portion; and a proximal projecting portion protruding away from the cap in a proximal direction from a proximal side of the seal body portion.

7. The syringe assembly according to claim 6, wherein the cap includes a through hole passing through a central part of the cap, the distal projecting portion of the seal member being located in the through hole of the cap.

8. The syringe assembly according to claim 6, wherein the proximal projecting portion of the seal member passes through the distal opening of the distal nozzle portion and is positioned in the lumen of the syringe.

9. A syringe assembly comprising:
a syringe that includes a distal nozzle portion, the syringe including a lumen extending throughout the distal nozzle portion and opening to a distal opening at a distal end of the distal nozzle portion, the distal end of the distal nozzle portion including a sealing surface that surrounds the distal opening at the distal end of the distal nozzle portion;
a cap fixed to the distal nozzle portion, the cap including a pressing surface that faces toward the sealing surface at the distal end of the distal nozzle portion;
a seal member made of elastic material and positioned between the sealing surface of the distal nozzle portion and the pressing surface of the cap;
the seal member including both a proximal end surface in direct contact with a contact portion of the sealing surface of the distal nozzle portion and a distal end surface in direct contact with a contact portion of the pressing surface of the cap so that the seal member closes and seals the distal opening at the distal end of the distal nozzle portion;
the seal member being pressed and held in an axial direction in a pressing space between the pressing surface and the sealing surface;
the contact portion of the pressing surface and the contact portion of the sealing surface being configured so that a first straight line distance between the contact portion of the pressing surface and the contact portion of the sealing surface is less than a second straight line distance between the contact portion of the pressing surface and the contact portion of the sealing surface; and
the first straight line distance and the second straight line distance both being parallel to the central axis of the distal nozzle portion, the first straight line distance being spaced from the second straight line distance, the first straight line distance being located closer to the central axis of the distal nozzle portion than the second straight line distance.

10. The syringe assembly according to claim 9, wherein the contact portion of the sealing surface of the distal nozzle portion includes an annular protrusion projecting toward the contact portion of the pressing surface of the cap, the annular protrusion being spaced from an outer periphery of the contact portion of the sealing surface toward the distal opening.

11. The syringe assembly according to claim 9, wherein the contact portion of the sealing surface of the distal nozzle portion is inclined to gradually protrude toward the contact portion of the pressing surface of the cap from an outer peripheral side of the contact portion of the sealing surface to an inner peripheral side of the contact portion of the sealing surface.

12. The syringe assembly according to claim 9, wherein the contact portion of the pressing surface of the cap includes an annular protrusion projecting toward the contact portion of the sealing surface of the distal nozzle portion, the annular protrusion being spaced inwardly from an outer periphery of the contact portion of the pressing surface.

13. The syringe assembly according to claim 9, wherein the contact portion of the pressing surface of the cap is inclined to gradually protrude toward the contact portion of the sealing surface of the distal nozzle portion from an outer peripheral side of the contact portion of the pressing surface to an inner peripheral side of the contact portion of the pressing surface.

14. The syringe assembly according to claim 9, wherein the seal member comprises: a plate-shaped seal body portion that is a central portion of the seal member in a thickness direction of the seal member; a distal projecting portion protruding in a distal direction from a distal side of the seal body portion; and a proximal projecting portion protruding away from the cap in a proximal direction from a proximal side of the seal body portion.

15. The syringe assembly according to claim 14, wherein the cap includes a through hole passing through a central part of the cap, the distal projecting portion of the seal member being located in the through hole of the cap.

16. The syringe assembly according to claim 14, wherein the proximal projecting portion of the seal member passes through the distal opening of the distal nozzle portion and is positioned in the lumen of the syringe.

17. The syringe assembly according to claim 9, wherein the cap is fixed to the distal nozzle portion by way of claw portions on the cap that each engage a respective engagement projecting portion on the syringe.

18. A liquid-medicine administration device comprising:
a syringe assembly comprising: a syringe that includes a distal nozzle portion and a lumen; a cap fixed to the distal nozzle portion; and a seal member arranged between the cap and the distal nozzle portion and made of an elastic body that seals the distal nozzle portion, the seal member including a distal end facing towards the cap and a proximal end facing away from the cap, the cap including a pressing surface that is in surface contact with a distal end of the seal member and presses the seal member toward a proximal side, the distal nozzle portion including a distal end at which is located a sealing surface, the sealing surface of the distal nozzle portion opposing the pressing surface and being in surface contact with the proximal end of the seal member, the distal nozzle portion including a distal opening on an inner peripheral side of the sealing surface, the distal opening of the distal nozzle portion being in communication with the lumen in the syringe, the seal member closing the distal opening and being pressed and held in an axial direction in a pressing space over a whole region of an opposing portion between the pressing surface and the sealing surface, and a gap between the pressing surface and the sealing surface in the pressing space being narrowest on an inner peripheral side;
a gasket slidably positioned inside the syringe;
a plunger assembly configured to press the gasket in a distal direction to slidably move the gasket in the distal direction inside the syringe; and
a drive mechanism driving the plunger assembly.

19. The liquid-medicine administration device according to claim 18, further comprising a chassis in which is supported the syringe assembly, the plunger assembly, and the drive mechanism.

20. The liquid-medicine administration device according to claim 18, further comprising a housing having an interior in which is housed a chassis;
the syringe assembly, the plunger assembly and the drive mechanism being supported on the chassis that is housed in the interior of the housing;
the distal nozzle portion of the syringe assembly protruding outside the housing, the cap being positioned outside the housing.
